(19) United States
(12) Patent Application Publication
(10) Pub. No.: US 2004/0043088 A1
(43) Pub. Date: Mar. 4, 2004

(54) Process for producing germ extract

(76) Inventors: Yaeta Endo, Ehime (JP); Masaharu Yamamoto, Shiga (JP)

Correspondence Address: Luke A Kilyk, Kilyk & Bowersox, 53 A East Lee Street, Warrenton, VA, 20186, US

(21) Appl. No.: 10/416,089

(22) PCT Filed: Nov. 8, 2001
(86) PCT No.: PCT/JP01/09778

(30) Foreign Application Priority Data
Nov. 8, 2000 (JP) 2000340576
Nov. 9, 2000 (JP) 2000341398

(51) Int. Cl. A01H 1/00; C12N 15/82; A01N 65/00

(52) U.S. Cl. 424/776; 800/278

(57) ABSTRACT

The present invention provides a process for producing embryo extract material characterized in comprising the steps where material plant seeds are mildly milled for removal of endsperm of plant seeds, from the milled product of the said material plant seeds are recovered fractions passing through 1.00˜0.45 mm sieves under shaking condition, the periderm of seeds is eliminated by winnowing, suspension supernatant in water or in aqueous solution free from organic solvent is recovered (flotation) and washing with water free from organic solvent or aqueous solution is carried out; embryo extract material produced by the said process; embryo extract obtained from the said embryo extract material; and a method for protein synthesis with the said embryo extract.

This application claims priority form Japanese Patent Application No.2000/340,576 and No.2000/341,398, which are incorporated herein by reference.

### TECHNICAL FIELD

The present invention relates to a material for embryo extract for a cell-free protein synthesis and a process for producing the same; to an embryo extract obtained from the said material and a process for producing the same; and to a method for protein synthesis using the said embryo extract. Its characteristic is to provide an efficient improved method for an art of removal of the self protein synthetic reaction inhibiting the mechanism being operated during the damage of living tissues and cells or, in other words, the self protein synthesis reaction destruction mechanism as a self-defense mechanism against diseases which is physiologically equipped and also for an art of neutralization of protein synthesis reaction inhibiting the activity induced as a result of milling of embryos whereby there is provided embryo extract for a cell-free protein synthesis having a high synthetic efficiency at a very low cost.

### BACKGROUND OF THE INVENTION

As the completion of the genome project is coming near, center of the studying matters has been quickly developed from analysis of gene structure to analysis of gene function. It is believed that protein in cells does not function solely as it is but its function is expressed as a result of interaction by cooperation with various kinds of protein factors, nucleic acids, low-molecular species, cell membrane component, etc. and, further, biological function is carried out by the said interaction as a whole. One of the central matters for the post-genome project is to analyze the relation between structure and function of various kinds of protein factor complexes. From the studies on fundamental biology, etc. including structural biology and biochemistry, it is expected that the fruit obtained therefrom provides very important findings for clarification of relation between gene translation product and cause for diseases in a medical field as its application and also for development of pharmaceuticals in broad fields.

As an in vitro method for conducting the protein synthesis reaction which efficiently proceeds in cells, there have been briskly carried out the studies for the so-called cell-free protein synthesis method where components including ribosome which are protein translation apparatuses equipped in cells are extracted from living body and protein is synthesized in vitro by addition of translation template, amino acids as substrates, energy source, various kinds of ion, buffer and other effective factors to the extract (Japanese Patent Laid-Open Nos. 06/98,790, 06/225,783, 07/194, 09/291 and 07/147,992).

Escherichia coli, wheat embryo, rabbit reticulocyte, etc. have been used for the reaction system for the cell-free protein synthesis or, in other words, for the preparation of cell extract or living tissue extract for protein synthesis. The cell-free protein synthesis system has properties which are equal to living cells in two terms of rate of peptide synthesis and precision in translation reaction and further has an advantage that neither complicated chemical reaction step nor troublesome cell incubation step is necessary whereby its practical system has been developed up to now. Usually however, in the cell extract from cells of living bodies, its protein synthesizing ability is very unstable whereby its protein synthesis efficiency is low and, in addition, lowering of quality of the cell extract during preservation is also significant. Therefore, amount of the synthesized product obtained by the cell-free protein synthesis system is so small that it can be detected by labeling with radioisotope, etc. whereby it is unable to be used as a practical means of production for protein.

As the solution for the disadvantages in the conventional cell-free protein synthesis system, the present inventors have previously provided (1) a cell extract preparation for a cell-free protein synthesis and a cell-free protein synthesis system (WO 00/68412) and (2) template molecules equipped with multi-purpose property or highly efficient function and a cell-free protein synthesis system utilizing the same (WO 01/27260).

Significance of more complete removal of endsperm from plant seeds as the same theme in the present invention is disclosed in the above (1). However, the method disclosed therein is a method using an organic solvent and, further, selection of embryos adopts a method by naked eye whereby those are industrially very limited as a providing means for embryo extract material. Accordingly, there has been a demand for providing a method for the manufacture of embryo extract material better than the conventional methods in an industrial view.

### DISCLOSURE OF THE INVENTION

An embodiment of the present invention is a process for producing an embryo extract material characterized in comprising the steps where material plant seeds are mildly milled for removal of endsperm of plant seeds, fractions having specific particles size are fractionated from the milled product of the said material plant seeds, fractionation by means of difference in weight is carried out, fractionation by means of difference in specific gravity is carried out and the surface part is subjected to a contacting treatment with water to fractionate.

An embodiment of the present invention is a process for producing an embryo extract material which is characterized in comprising the steps where material plant seeds are mildly milled for removal of endsperm of plant seeds, fractions having specific particles size are fractionated from the milled product of the said material plant seeds, fractionation by means of difference in weights is carried out, fractionation by means of difference in specific gravities without the use of organic solvent is carried out and the surface part is subjected to a contacting treatment with water to fractionate.

An embodiment of the present invention is a process for producing an embryo extract material which is characterized in comprising the steps where material plant seeds are mildly milled for removal of endsperm of plant seeds, fractions passing through 1.00˜0.45 mm sieves are recovered under a shaking condition from the milled product of the said material plant seeds, the periderm of seeds is eliminated by winnowing, suspension supernatant in water or in aqueous solution free from organic solvent are recovered (flotation) and washing with water free from organic solvent or aqueous solution is carried out.

An embodiment of the present invention is a process for producing an embryo extract material characterized in comprising the steps where material plant seeds are mildly milled for removal of endsperm of plant seeds, fractions passing through 1.00˜0.71 mm sieves are recovered under a shaking condition from the milled product of the said material plant seeds, the periderm of seeds is eliminated by winnowing, suspension supernatant in water or in aqueous solution free from organic solvent is recovered (flotation) and washing with water free from organic solvent or aqueous solution is carried out.

An embodiment of the present invention is a process for producing an embryo extract material which is characterized in comprising the steps where material plant seeds are mildly milled for removal of endsperm of plant seeds selected from a group consisting of wheat, barley, rice, corn and spinach, fractions passing through 1.00˜0.71 mm sieves are recovered under a shaking condition from the milled product of the said material plant seeds, the periderm of seeds is eliminated by winnowing, suspension supernatant in water or in aqueous solution free from organic solvent is recovered (flotation) and washing with water free from organic solvent or aqueous solution is carried out.

An embodiment of the present invention is a process for producing an embryo extract material which is characterized in comprising the steps where material plant seeds are mildly milled for removal of endsperm of plant seeds selected from a group consisting of wheat, barley, rice, corn and spinach, fractions passing through 1.00˜0.71 mm sieves are recovered under a shaking condition from the milled product of the said material plant seeds, the periderm of seeds is eliminated by winnowing, suspension supernatant in water or in aqueous solution free from organic solvent is recovered (flotation), washing with water free from organic solvent or aqueous solution is carried out and washed embryos are subjected to a supersonic treatment in an aqueous solution containing surface-active agent and/or formycin 5′-phosphate.

An embodiment of the present invention is an embryo extract material obtained by the above-mentioned producing process.

An embodiment of the present invention is a process for producing an embryo extract characterized in using the embryo extract material obtained by the above-mentioned producing process.

An embodiment of the present invention is an embryo extract material obtained by the above-mentioned process for producing an embryo extract.

An embodiment of the present invention is a method for protein synthesis of with an embryo extract material obtained by the above-mentioned process for producing an embryo extract.

### BRIEF DESCRIPTION OF THE DRAWINGS

FIG. 1 shows a multilayered method.

FIG. 2 is a picture showing the result where green fluorescent protein (GFP) synthesized by a cell-free protein synthesis system by a multilayered method using a wheat embryo extract obtained by the process of the present invention or using a wheat embryo extract obtained by the convention method is detected by an SDS electrophoresis. Lanes \mathring{1} and \mathring{8} are the result of electrophoresis of molecular weight marker (LMW marker) (manufactured by Amersham-Pharmacia); lanes \mathring{2} and \mathring{7} are that of 0.50 micro g and 0.25 micro g, respectively, of recombinant GFP (rGFP) (manufactured by Wako Pure Chemical); lane \mathring{3} is that of 1 micro 1 of reaction solution upon reaction for 0 hour using a wheat embryo extract obtained by the conventional method; lane \mathring{4} is that of 1 micro 1 of reaction solution upon reaction for 0 hour using a wheat embryo extract obtained by the method of the present invention; lane \mathring{5} is that of 1 micro 1 of reaction solution after the reaction for 17 hours with a wheat embryo extract obtained by the conventional method; and lane \mathring{6} is that of 1 micro 1 of reaction solution after the reaction for 17 hours with a wheat embryo extract obtained by the method of the present invention.

FIG. 3 shows a dialytic method.

FIG. 4 shows pictures of the results where green fluorescent protein synthesized by a cell-free protein synthesis system by a dialytic method is detected by an SDS electrophoresis. (a) shows the result when the wheat embryo extract obtained by the conventional method is used for cell-free protein synthesis while (b) shows the result when the wheat embryo extract obtained by the method of the present invention is used therefor. Lanes \mathring{1} and \mathring{8} in each of them are the result of electrophoresis of a molecular weight marker (LMW marker) (manufactured by Amersham-Pharmacia); lane \mathring{2} is that of 0.50 micro g of recombinant GFP (rGFP) (manufactured by Wako Pure Chemicals); and lanes (0 to 0 are that of ⅓ micro 1 of reaction solution after the reaction was conducted for 0, 24, 48, 72 and 96 hours, respectively using the wheat embryo extract obtained by each of the methods.

### BEST MODE FOR CARRYING OUT THE INVENTION

The embryo extract according to the present invention is prepared using embryos where endsperm part is removed almost completely as material. In order to prepare the embryo extract according to the present invention, it is necessary that, at first, endsperm containing the intrinsic specific inhibitor is removed almost completely to purity embryos. In the present invention, the embryo extract free from endsperm is an embryo extract where endsperm part is removed to such an extent that ribosome is not substantially deadenylated. Incidentally, the extent that ribosome is not substantially deadenylated means the deadehyration rate of ribosome is less than 7% or, preferably, 1% or less.

With regard to plant seeds usable in the present invention, seeds of plant selected from wheat, barley, rice, corn and spinach are usually exemplified. Among them, appropriate plant seeds are wheat, barley and rice and the particularly preferred one is wheat.

The plant seeds are firstly milled. It is preferred that the milling is conducted mildly. For example, the milling is carried out in such a manner that seeds are added to the mill at the rate of 50 g˜1 kg per minute and milled mildly with a revolution of 5,000˜10,000 rpm. The milled seeds are fractionated depending upon their particle size. For the fractionation, sieves may be simply used although there is no particular limitation so far as a fractionation method depending upon the particle size being utilizable by persons skilled in the art is used. When sieves are used, fractions passing through 1.00˜0.45 mm sieves are recovered. Preferably, fractions passing through 1.00˜0.71 mm sieves are recovered. As a result of such a treatment, a crude embryo fraction is collected.

The crude embryo fraction contains a periderm component having a size of 1.00 mm or less as well and, therefore, eliminating the periderm component is further necessary. Since the periderm component is light as compared with embryo, it can be eliminated preferably by means of fractionation depending upon the difference in weight. There is no limitation for a eliminating method so far as it is a method on the basis of difference in weight and is utilizable by persons skilled in the art. For example, the periderm component is blown off by wind on a sieve having a mesh size of 0.50 mm or 0.45 mm whereupon the periderm component can be fractionated and eliminated (winnowing). As a result of such a treatment, there is obtained a crude embryo fraction in a size of 0.45˜1.00 mm or, preferably, 0.71˜0.85 mm.

After that, the crude embryo fraction is further fractionated preferably depending upon the difference in specific gravity. With regard to an embodiment therefor, a fractionation on the basis of difference in flotage may be exemplified. With regard to the fractionation on the basis of difference in flotage, floatation is usually recommended. In the floatation, crude embryo fraction is dipped in water or in aqueous solution, the substance which is floated on the supernatant liquid within short time is recovered and is selected from a precipitated substance. Preferably, the crude embryo fraction which is once sedimented in water or in aqueous solution is floated, then oscillation is applied so that the contaminated the periderm component and trash are firstly precipitated and the supernatant liquid fraction is recovered. It is preferred that the floatation is carried out repeatedly although the present invention is not particularly limited thereto. More preferably, the floatation is repeated for two to three times.

Floatation is carried out using water or aqueous solution free from organic solvent. With regard to the aqueous solution, there is no particular limitation so far as the protein synthetic ability of embryo is not lowered and only embryos are floated on the water surface while contaminants such as the periderm component are able to sink. It is preferred that water or aqueous solution used is in low temperature and that the temperature is lower than the temperature where the embryo does not start its activity. More preferred temperature is about 5° C. or lower. For example, ice-cooled water is preferably used. Water may be either tap water or may be distilled water or water where distilled water is further deionized. It is also possible that a protease inhibitor is added to water or to aqueous solution.

As mentioned above, after fractionation on the basis of particle size, difference in weight and difference in specific gravity, the contaminant components remaining on the surface part of the crude embryo fraction are fractionated by a contacting treatment with water or with aqueous solution. One of the means for the contacting treatment with water or aqueous solution is washing for example or a rubbing treatment by water or aqueous solution. As a result of such a treatment, the remained endsperm component, for example, is easily dissolved into water or aqueous solution. With regard to the contacting treatment with water or aqueous solution, there is exemplified a washing step of the crude embryo fraction with a washing solution free from organic solvent. For washing, it is effective to add physical shock (physical contacting treatment) such as rubbing, collision and/or stirring. It is preferred that washing is well carried out together with exchanging the washing solution until the washing solution does not become turbid any more. Water or aqueous solution is used as the washing solution. For example, the crude embryo fraction obtained by flotation is wrapped with layered gauze or the like and subjected to hand-washing in a washing solution together with exchange of the washing solution. It is also possible, for example, that the crude embryo fraction is suspended in a washing solution and subjected to a physical shock such as stirring and the crude embryo fraction was separated from the turbid washing solution using known separating means such as filtration. In that case, it is also possible that washing operation and separating operation are carried out in the same container. There is no particular limitation for the aqueous solution so far as it does not lower the protein synthetic ability of embryo. The washing solution is preferably in low temperature and the temperature is preferably the temperature where the embryo does not start its activity. More preferred temperature is about 5° C. or lower. For example, ice-cooled water is preferably used. With regard to water, distilled water or that which is further deionized or sterilized water is preferred.

If desired, the crude embryo fraction washed as such may be further subjected to an ultrasonic wave treatment in aqueous solution containing surface-active agent and/or formycin 5′-phosphate. The ultrasonic treatment may be carried out according to a known means and, although the treating time of several minutes within 10 minutes is sufficient, it may vary depending upon the treating amount. It is preferred that the ultrasonic treatment is carried out repeatedly although there is no particular limitation therefor. More preferably, it is repeated for two or three times. The use of surface-active agent is also in accordance with the known means. For example, with regard to the surface-active agent, there may be used Nonidet P-40 (NP-40) and IGEPAL CA-630 (manufactured by Sigma; code: I 3021) and it may be used in a concentration of 0.01˜0.90%, preferably 0.03˜0.70% or, more preferably, 0.05˜0.50%. After the ultrasonic treatment by aqueous solution containing surface-active agent and/or formycin 5′-phosphate, it is preferred to wash with water or aqueous solution free from them for the removal of surface-active agent and/or formycin 5′-phosphate. With regard to the aqueous solution for the treatment, there is no particular limitation so far as it does not lower the protein synthetic ability of the embryo. It is preferred that water or aqueous solution is in low temperature and the temperature is preferably to be the temperature where embryo does not start its activity. More preferred temperature is about 5° C. or lower. For example, ice-cooled water is preferably used. With regard to water, it is more preferred to use distilled further deionized or to use sterilized water.

The embryo fraction recovered after each of the above-mentioned treatments was pure product being beautiful in its appearance and was nearly the same as the embryo material obtained by the conventional treatment with organic solvent and selection by naked eye. Thus, the said pure product was used as an embryo extract material.

Preparation of the embryo extract is carried out by a conventional method using the above-mentioned material. For example, that may be carried out in accordance with an already-reported method (Erickson, A. H., et al., (1996) Meth. in Enzymol., 96, 38-50). The embryo extract may be prepared as a liquid or the embryo extract liquid may be made into solid such as powder by a known method such as freeze-drying.

The embryo extract prepared as such may be used in the so-called cell-free protein synthesis system. Protein synthesis by the cell-free protein synthesis system according to the present invention may be carried out by the same method as in the conventional one except that, as an embryo extract liquid, there is used embryo extract prepared from embryo extract material free from endsperm. The above-mentioned cell-free protein synthesis system may be any of known ones and there may be exemplified a batch method and a synthetic method where amino acid and energy source are continuously supplied (hereinafter, referred to as a continuously supplying synthetic method) such as a continuous cell-free protein synthesis system by Spirin, et al. In a batch method, the reaction may stop when protein synthesis is carried out for long time but, in the latter continuously supplying synthetic method, the reaction is able to be maintained for long time whereby it is possible to promote the efficiency. Further, in the protein synthesis by the continuously supplying synthetic method, a dialytic method is usable together. For example, it is possible to synthesize protein continuously and in large quantities according to an ultrafilter membrane dialytic synthetic method where the embryo extract of the present invention is used as an inner solution for dialysis while a mixed liquid containing energy source and amino acid is used as an outer solution for dialysis. Here, with regard to the energy source, there may be exemplified adenosine triphosphate (ATP), guanosine triphosphate (GTP) and creatine phosphate while, with regard to amino acid, there may be exemplified 20 kinds of L-amino acids. To be more specific, a method according to that disclosed in the Japanese Patent Laid-Open No. 2000/236,896 may be carried out.

In the Examples of the present invention, there are shown embryo extract material using wheat as an example and manufacture of embryo extract using the said material and a process for the portein synthesis using the said embryo extract although the present invention is not limited thereto. For the purpose of removal of endsperm-derived substances which are contaminated in the preparation of embryo extract material, the present inventors have investigated a method for washing the crude embryo fraction separated from wheat seeds using a mill and, as a result, contamination of endsperm-derived substances in embryo fraction has now been removed almost completely without the use of organic solvent and without the necessity of selection by naked eye. When there were attempted a cell-free protein synthesis of a multilayered type and cell-free protein synthesis of a dialysis type using such pure embryos and using mRNA coding for green fluorescent protein (hereinafter, abbreviated as GFP) as a model template, there was achieved the same synthetic efficiency as compared with the case using embryos obtained by a method using organic solvent as disclosed by the present inventors in the Japanese Patent Laid-Open No. 2000/236,896 already.

### EXAMPLES

As hereunder, the present invention will now be illustrated in more detail by way of Examples and Experimental Examples although the following Examples and Experimental Examples should be considered merely as a hint for knowing the specific recognition for the present invention and the scope of the present invention is never limited by such examples.

Firstly, compositions of the buffers used in the following Examples and Experimental Examples will be shown below.

| Buffer A: | | |
|---|---|---|
| 160 | mM | HEPES-KON, pH 7.6 |
| 400 | mM | Potassium acetate (KOAc) |
| 4 | mM | Magnesium acetate [Mg(OAc)₂] |
| 8 | mM | Calcium chloride |
| 16 | mM | Dithiothreitol (DTT) |
| 1.2 | mM | AAs |
| | | (a mixture of 20 kinds of L-amino acid) |
| Buffer B: | | |
| 40 | mM | HEPES-KOH, pH 7.6 |
| 100 | mM | Potassium acetate |
| 5 | mM | Magnesium acetate |
| 4 | mM | DTT |
| 0.3 | mM | AAs |
| Buffer C: | | |
| 31.3 | mM | HEPES-KOH, pH 7.6 |
| 93 | mM | Potassium acetate |
| 2.67 | mM | Magnesium acetate |
| 2.1 | mM | Dithiothreitol |
| 0.3 | mM | AAs |
| 1.2 | mM | ATP |
| 0.257 | mM | GTP |
| 1 micro M | | E-64 |
| 0.005% | | Sodium azide |
| 0.41 | mM | Spermidine |
| 16 | mM | Creatine phosphate |

### Example 1

Purification of Wheat Embryo

Chihoku wheat seeds made in Hokkaido (non-disinfected) (5 kg) were added to a mill (Rotor Speed Mill Pulverizette, type 14; manufactured by Fritsch) continuously at the rate of 100 grams per minute and the seeds were mildly milled at 8,000 rpm. The milled seeds were applied to a sieve shaker (Analysette 3; an electromagnetic sieve shaker for experiments manufactured by Fritsch) and fractions passing through 1.00˜0.71 mm sieves are recovered.

Winnowing was carried out at room temperature using a test rice huller manufactured by K. K. Satake (THU 35B) where improvement was done that a cover was attached on the upper part of a cyclone. A valve for air volume of the said rice huller was opened to an extent of 0.5˜1.0 mm from the closed state and the cover on the upper part of the cyclone was opened to an extent of 15 mm whereupon flow volume was adjusted. The milled seeds of 1.00˜0.71 mm recovered from the above sieve shaker were filled in a hopper of the rice huller as a sample, the knob adjusting the flow rate was closed to such an extent that embryos were able to pass through (gap: about 1.0 mm) and a roll gap knob was similarly adjusted to such an extent that embryos were able to pass through (gap: 1.0˜1.5 mm). A shutter was gradually opened to drop the sample whereupon winnowing was conducted. The sample mostly comprising embryo wherefrom periderm was removed came to an immature grain container and a container for selected particle size and that was used as a crude embryo fraction.

The resulting crude embryo fraction (about 26 grams) was placed in a sieve (mesh size: 0.50˜0.45 mm), gently sunk in a container to which a lot of ice-cooled water (about 0° C.) was added, all of the crude embryo fraction was once sunk in water, taken out from water together with the sieve and gently sunk together with the sieve Whereupon floatation was carried out. Since embryos were floated on the supernatant part by means of floatation, a little oscillation was applied to the sieve so that contaminants such as trash were firstly sunk and embryos in the remaining supernatant fraction were recovered. After the flotation operation was repeated for five times, the recovered embryo fractions were taken on paper towels to remove moisture. The embryo fraction was subjected to a floatation step repeatedly as same as above and moisture was removed on paper towels. As a result, about 14.25 g of embryos (crude embryo fraction) were obtained. The crude embryo fraction (3 g) was wrapped with four layers of gauze and well washed in a hand-washing sense in a 300-ml beaker using 300 ml of ice-cooled (about 0° C.) pure water (hereinafter, may be referred to as ddw) of resistance of 18 omega prepared by deionization of distilled water by filtering through Milli Q (manufactured by Millipore) as a washing solution until the washing solution did not become turbid where the washing solution was exchanged for ten times or more. After that, it was washed for 5 minutes in 300 ml of 0.05% IGEPAL CA-630 solution using an ultrasonic washer (manufactured by Yamato; Branson Model 2210 Sonicator). After that, an ultrasonic washing was carried out where the washing solution was exchanged to 300 ml of ddw and then more 300 ml of ddw were exchanged to conduct the ultrasonic washing once again. The wheat embryos were taken out from the gauze onto a Buchner funnel and subjected to filtration and washing with 500 ml of ddw and moisture was removed therefrom to give pure wheat embryos.

### Example 2

Preparation of Wheat Embryo Extract

Preparation of wheat embryo extract was carried out in a refrigerator of 4° C. Embryos obtained in Example 1 wherefrom moisture was well removed were frozen with liquid nitrogen and well milled until powdery state keeping the temperature at not higher than −150° C. To the resulting powder was added 1 ml of the buffer A and the temperature was gradually raised together with well grinding. The embryo powder which became thick was filled in a centrifugal tube and centrifuged at 30,000′ g for 15 minutes and the resulting supernatant fraction was recovered, added to Sephadex G-25 column which was previously equilibrated with the buffer B and centrifuged at 3,000 rpm for 5 minutes to conduct a gel filtration. The liquid recovered by the gel filtration was further centrifuged at 30,000′ g for 12 minutes and the supernatant fraction wherefrom the top liquid was removed was recovered, separately placed and preserved by freezing in liquid nitrogen. The recovered liquid amount of 1.2 ml and O.D._{260 nm} of the said recovered liquid was 220.5.

### Experimental Example 1

Cell-Free Protein Synthesis by Multilayered Method

Synthesis of GFP was carried out by a multilayered method using the embryo extract obtained in Example 2 and compared with the embryo extract liquid (Proteios; manufactured by Toyobo) obtained by a conventional method (Japanese Patent Laid-Open No. 2000/236,896).

With regard to GFP, its expression was confirmed by fluorescent coloration by naked eye after several hours from the initiation of the reaction.

With regard to the experimental operation, there were carried out preparation of mRNA, preparation of reaction solution, synthetic reaction of GFP and confirmation of the reaction according to the following procedures.

1) Preparation of mRNA

A reaction solution [80 mM of HEPES-KOH, pH 7.6; 16 mM of magnesium acetate; 10 mM of dithiothreitol; 2 mM of spermidine; 2.5 mM of 4NTPs (four kinds of nucleotide triphosphates); 1.16 U/micro 1 of RNase inhibitor; 0.1 micro g/micro 1 of DNA (plasmid GFP) and 3 U/micro 1 of SP6 RNA polymerase] was prepared and made to react at 37° C. for 3 hours.

After that, it was subjected to gel filtration using a G-25 spin column which was previously equilibrated with the buffer C. The filtered solution was preserved in ice water until immediately before use. Concentration of mRNA obtained as such was 1.2˜1.6 micro g/micro 1.

2) Preparation of the Reaction Solution

Each of the embryo extract liquid (Proteios; manufactured by Toyobo) obtained by the conventional method (Japanese Patent Laid-Open No. 2000/236,896) and the embryo extract liquid obtained by the method of the present invention was made into a uniform concentration (O.D._{260 nm}=200) using the buffer B and used. To a solution containing the embryo extract liquid (60.7% of the extract liquid; 2.82 U/micro 1 of RNase inhibitor; 0.005% of sodium azide; 0.2 micro g/micro 1 of tRNA; 1 micro M of E-64; 42 mM of potassium acetate; 1.03 micro g/micro 1 of creatine kinase; 126 micro M of AAs; 1.21 mM of ATP; 0.267 mM of GTP; 16.1 mM of creatine phosphate; and 0.455 mM of spermidine) was added 2-fold volume of the previously prepared mRNA solution (which was previously prepared by diluting to 0.8 micro g/micro 1 using the buffer C) whereupon a protein synthesis reaction solution was prepared.

3) Initiation of the Reaction

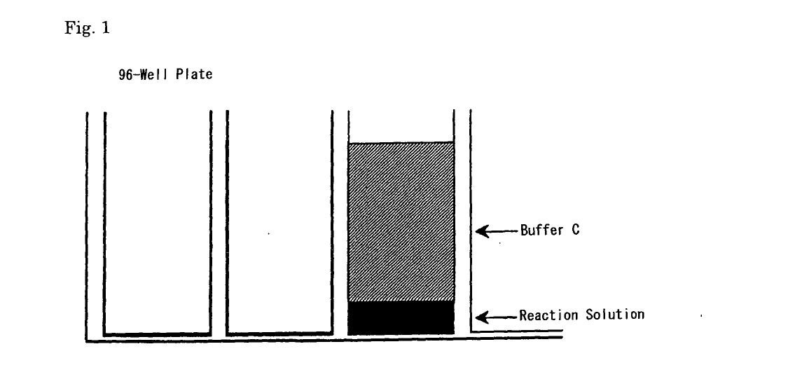

A protein synthesis reaction was carried out by a multilayered method using a 96-well plate as a reactor. The buffer C (250 micro 1) was added per one well, 50 micro 1 of the reaction solution prepared in the above 2) were gently added so as to form a layer thereunder as shown in FIG. 1 and the reaction was conducted at 26° C. for 17 hours.

4) Confirmation of Protein Synthetic Effect

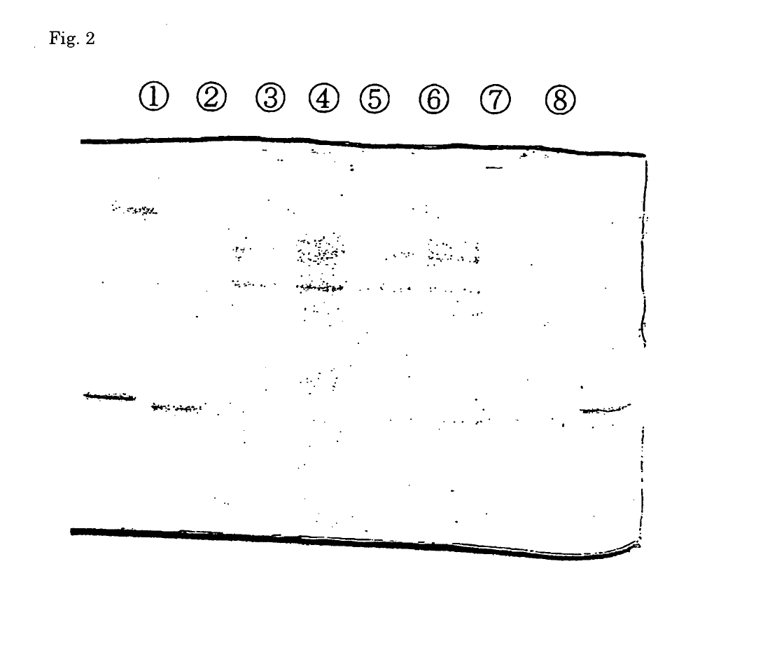

In order to confirm the reaction product, an SDS electrophoresis was carried out for 130 minutes at 30 mA using 15% acrylamide gel (FIG. 2). As a result, as compared with the embryo extract liquid obtained by the conventional method (Japanese Patent Laid-Open No. 2000/236,896) (lane \mathring{5} in the drawing), the embryo extract liquid obtained by the method of the present invention (lane \mathring{6} in the drawing) was confirmed to have the same synthetic ability.

### Experimental Example 2

Cell-Free Protein Synthesis by a Dialysis Method

Each of the embryo extract liquid obtained by the method of the present invention and the embryo extract liquid obtained by the conventional method (Japanese Patent Laid-Open No. 2000/236,896) (Proteios; manufactured by Toyobo) was used to conduct the synthesis of GFP by a dialysis method.

1) Preparation of mRNA

It was prepared by the same manner as in Experimental Example 1.

2) Preparation of the Reaction Solution

Each of the embryo extract liquid (Proteios; manufactured by Toyobo) obtained by the conventional method (Japanese Patent Laid-Open No. 2000/236,896) and the embryo extract liquid obtained by the method of the present invention was made into a uniform concentration (O.D._{260 nm}=200) using the buffer B and used. To a solution containing the embryo extract liquid (60.7% of the extract liquid; 2.82 U/micro 1 of RNase inhibitor; 0.005% of sodium azide; 0.2 micro g/micro 1 of tRNA; 1 micro M of E-64; 42 mM of potassium acetate; 1.03 micro g/micro 1 of creatine kinase; 126 micro M of AAs; 405 micro M of ATP; 89.1 micro M of GTP; 5.37 mM of creatine phosphate; and 152 mM of spermidine) was added 2-fold volume of the previously prepared mRNA solution (1.464 micro g/micro 1) whereupon a protein synthesis reaction solution was prepared.

3) Dialysis Method Reaction

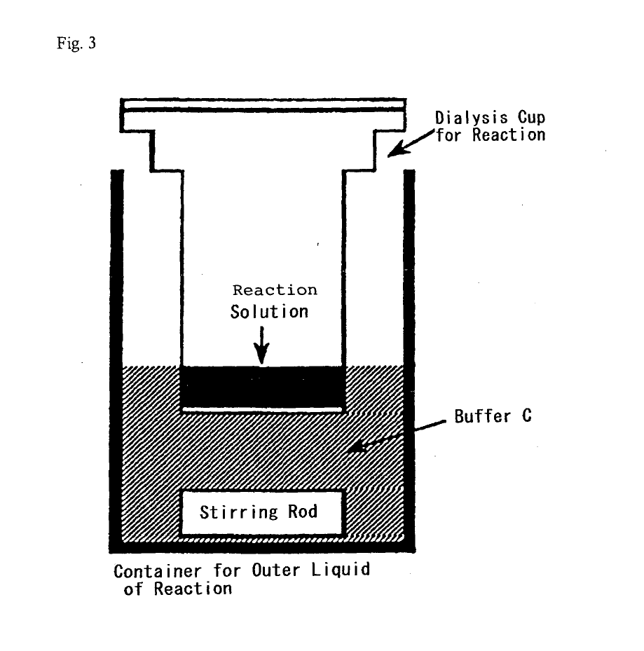

A container for outer liquid for the reaction (the upper part of the polypropylene container No. 3 manufactured by Inouchi Seieido was cut to an extent of 1 cm) and a stirring rod (manufactured by Inouchi Seieido; 10 mm′ 4 mm ø) were sterilized by boiling with ddw at 100° C. for 30 minutes, the stirring rod was placed in the container for outer liquid of the reaction wherefrom ddw was removed and inner part of the container and the stirring rod were washed with the buffer C for three times. After that, inner part and outer part of the dialysis cup for the reaction [Bio-Tech dialysis cup (MWCO 12,000) manufactured by Dauichi Kagaku Yakuhin] were washed with the buffer C for three times and set to the container for outer liquid of the reaction. The buffer C (1,700 micro 1) was added to the container for outer liquid for the reaction, then 150 micro 1 of the above-prepared reaction solution were added to the dialysis cup for the reaction and the reaction was carried out at 26° C. and 200 rpm together with stirring of the outer liquid for the reaction (FIG. 3). After 48 hours from the initiation of the reaction, 30 micro 1 of the above-prepared mRNA (43.92 micro g) dry-frozen after precipitating with ethanol were added to the reaction solution. The reaction was carried out for 24 hours, 48 hours, 72 hours or 96 hours.

4) Confirmation of the Protein Synthetic Effect

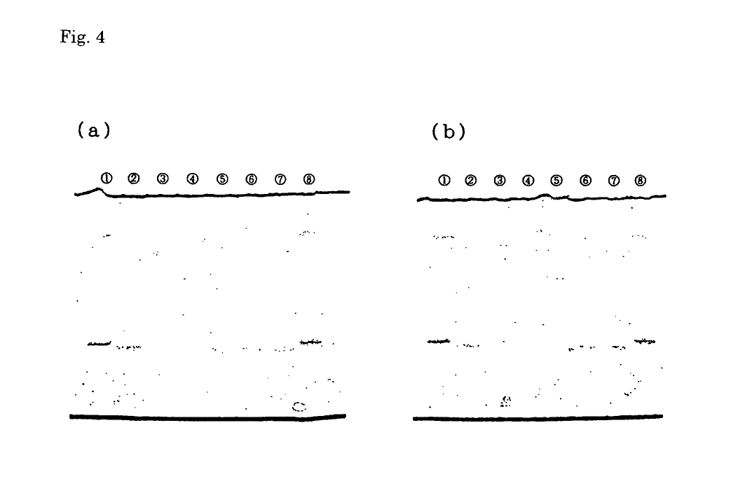

In order to confirm the reaction product, ⅓ micro 1 of each of the reaction solutions was subjected to an SDS electrophoresis for 130 minutes at 30 mA using 15% acrylamide gel [(a) and (b) of FIG. 4]. FIG. 4(a) shows the result where protein synthesis was carried out using the embryo extract liquid obtained by the conventional method while FIG. 4(b) shows the result where protein synthesis was carried out using the embryo extract liquid obtained by the method of the present invention. As compared with the embryo extract liquid obtained by the conventional method (Japanese Patent Laid-Open No. 2000/236,896), the embryo extract liquid obtained by the method of the present invention was confirmed to have the same continuous synthetic ability.

Industrial Applicability

In accordance with present invention, there are provided a very efficient and simple embryo extract material for a cell-free protein synthesis and a process for producing the embryo extract using said material. As compared with the conventional method, the method according to the present invention solves the environmental problems due to the use of organic solvent and the troublesomeness of selection by naked eye. The embryo extract for the cell-free protein synthesis provided by the method of the present invention is very useful for the mass production of protein in a cell-free system such as in a mass production of enzymes and antibodies and contributes in a broad area from the basic studies such as evolutionary molecule engineering to the development of pharmaceuticals.

1. A process for producing embryo extract material for removal of endsperm of plant seeds which is characterized in comprising the following steps:
(1) a step where material plant seeds are mildly milled,
(2) a step where fractions having specific particles size are fractionated from the milled product of the said material plant seeds,,
(3) a step where fractionation by means of difference in weight is carried out,
(4) a step where fractionation by means of difference in specific gravity is carried out and
(5) a step where the surface part is subjected to a contacting treatment with water to fractionate.

2. A process for producing embryo extract material for removal of endsperm of plant seeds which is characterized in comprising the following steps:
(1) a step where material plant seeds are mildly milled,
(2) a step where fractions having specific particles size are fractionated from the milled product of the said material plant seeds,
(3) a step where fractionation by means of difference in weight is carried out,
(4) a step where fractionation by means of difference in specific gravities using no organic solvent is carried out and
(5) a step where the surface part is subjected to a contacting treatment with water to fractionate.

3. A process for producing embryo extract material for removal of endsperm of plant seeds which is characterized in comprising the following steps:
(1) a step where material plant seeds are mildly milled,
(2) a step where fractions passing through 1.00˜0.45 mm sieves under a shaking condition are recovered from the milled product of the said material plant seeds,
(3) a step where the periderm of seeds is eliminated by winnowing,
(4) a step where suspension supernatant in water or in aqueous solution free from organic solvent is recovered (flotation) and
(5) a step where washing with water free from organic solvent or aqueous solution is carried out.

4. A process for producing embryo extract material steps for removal of endsperm of plant seeds which is characterized in comprising the following:
(1) a step where material plant seeds are mildly milled,
(2) a step where fractions passing through 1.00˜0.71 mm sieves under a shaking condition are recovered from the milled product of the said material plant seeds,
(3) a step where the periderm of seeds is eliminated by winnowing,
(4) a step where suspension supernatant in water or in aqueous solution free from organic solvent is recovered (flotation) and
(5) a step washing with water free from organic solvent or aqueous solution is carried out.

5. A process for producing embryo extract material for removal of endsperm of plant seeds selected from a group consisting of wheat, barley, rice, corn and spinach which is characterized in comprising the following steps:
(1) a step where material plant seeds are mildly milled,
(2) a step where fractions passing through 1.00˜0.71 mm sieves under a shaking condition are recovered from the milled product of the said material plant seeds,
(3) a step where the periderm of seeds is eliminated by winnowing,
(4) a step where suspension supernatant in water or in aqueous solution free from organic solvent is recovered (flotation) and
(5) a step where washing with water free from organic solvent or aqueous solution is carried out.

6. A process for producing embryo extract material for removal of endsperm of plant seeds selected from a group consisting of wheat, barley, rice, corn and spinach which is characterized in comprising the following steps:
(1) a step where material plant seeds are mildly milled,
(2) a step where fractions passing through 1.00˜0.71 mm sieves under a shaking condition are recovered from the milled product of the said material plant seeds,
(3) a step where the periderm of seeds is eliminated by winnowing,
(4) a step where suspension supernatant in water or in aqueous solution free from organic solvent is recovered (flotation),
(5) a step where washing with water free from organic solvent or aqueous solution is carried out and
(6) a step where the washed embryos are subjected to a supersonic treatment in an aqueous solution containing surface-active agent and/or formycin 5′-phosphate.

7. An embryo extract material obtained by the producing process mentioned in claim 6.

8. A process for producing an embryo extract characterized in using an embryo extract material obtained by the producing process mentioned in claim 6.

9. An embryo extract material obtained by the process for producing an embryo extract mentioned in claim 8.

10. A method for portein synthesis using an embryo extract material obtained by the process for producing an embryo extract mentioned in claim 8.
